(19) United States
(12) Patent Application Publication
(10) Pub. No.: US 2024/0382687 A1
(43) Pub. Date: Nov. 21, 2024

(54) Electronic module, drug delivery device and method for operating an electronic module

(71) Applicant: Sanofi, PARIS (FR)

(72) Inventor: Christopher Knorr, Frankfurt am Main (DE)

(21) Appl. No.: 18/692,388

(22) PCT Filed: Sep. 22, 2022
(86) PCT No.: PCT/EP2022/076281

§ 371 Date: Mar. 15, 2024

(30) Foreign Application Priority Data
Sep. 24, 2021 (EP) 21315171.5

(51) Int. Cl. A61M 5/315; G08C 17/02; G16H 20/17; G16H 40/67

(52) U.S. Cl. CPC A61M 5/31546; G16H 20/17; G16H 40/67; A61M 2205/3327; A61M 2205/3584; A61M 2205/52; A61M 2205/8206; G08C 17/02

(57) ABSTRACT

The present disclosure refers to an electronic module for a drug delivery device comprising a dose setting and drive mechanism, configured to perform a dose setting operation for setting a dose to be delivered by the drug delivery device and a dose delivery operation for delivering the set dose. The electronic module comprises at least one processor, a sensor arrangement connected to the at least one processor and operable to generate measurement data indicative of the dose setting operation and/or the dose delivery operation, a first communication unit with a wireless communication interface connected to the at least one processor and operable to establish an encrypted wireless communication with a second communication unit of another device and to transfer data to said other device, a memory for storing measurement data, and a power source connected to the at least one processor.

### CROSS REFERENCE TO RELATED APPLICATIONS

The present application is the national stage entry of International Patent Application No. PCT/EP2022/076281, filed on Sep. 22, 2022, and claims priority to Application No. EP 21315171.5, filed on Sep. 24, 2021, the disclosures of which are incorporated herein by reference.

### TECHNICAL FIELD

The present disclosure is generally directed to an electronic system, e.g., an electronic module, for a drug delivery device. The present disclosure further relates to a drug delivery device, which comprises such an electronic module, and a system comprising an electronic module as well as another device. Still further, the present disclosure is directed to a method for operating such an electronic module.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament.

There are basically two types of drug delivery devices: resettable devices (e.g., reusable) and non-resettable (e.g., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present disclosure is applicable for disposable and reusable devices.

For such devices, the functionality of recording doses that are dialed and/or delivered from the pen may be of value to a wide variety of device users as a memory aid or to support detailed logging of dose history. Thus, drug delivery devices using electronics are becoming increasingly popular in the pharmaceutical industry as well as for users or patients.

For example, a drug delivery device is known from EP 2 814 545 A1 comprising an electronic clip-on module. The clip-on module comprises a battery which powers a processor and further components controlled by the processor, like light-sources, a photometer, an acoustic sensor, an acoustical signal generator and a wireless unit, like a Bluetooth® transceiver configured to transmit and/or receive information to/from another device, e.g., a smart phone, in a wireless fashion.

Information, for example information gathered during dose setting and/or dose delivery, may be transmitted to another device for evaluation of said information by a physician and/or a user. This may be especially helpful if the drug delivery does not contain a display, if the display is not suitable for displaying said information or if additional software, which may be used on said other device, is needed in order to evaluate said information properly.

In addition, the transfer of information from the electronic module to another device can be helpful in order to reduce the memory usage of the electronic module and thus enable the design of an electronic module with a smaller memory, which may thereby consume less power.

Further, the electronic module and, if applied to a drug delivery device, said drug delivery device may be able to receive information from the other device, such as information regarding a firmware update.

In order to be able to exchange information using the same transmitting technology, such as Bluetooth, e.g., to transmit and/or receive information wirelessly over a certain distance, the drug delivery device and the other device need to establish wireless communication. However, such a pairing process is particularly vulnerable to unwanted attacks, e.g., so-called ‘man-in-the-middle-attacks’, wherein an unauthorized third party interferes in the pairing process and accesses confidential patient data. In particular, such an attack is possible due to the fact that during the pairing process, private information, such as a pairing key, is usually sent in an undirected manner within a transmission range, a so-called pairing range, with the aim that another device within this pairing range receives and responds to the transmitted information, e.g., exchanging pairing data, thereby pairing the two devices. After successful pairing the exchange of information or data may be protected against third parties, for example by encryption.

To improve the security of a wireless data transfer between two devices different methods are known. One method described in EP 1 997 233 B1 discloses the pairing of a drug delivery device with another device such as a remote controller adapted to transmit instructions or to receive and store data such as sensor data. In order to enable a secure data exchange data is classified according to certain security levels and corresponding means of communication, so that data with a certain security level is exchanged via certain means of communication, wherein the means of communication may have different ranges of communication.

Further, WO 2020/193090 A1 discloses, in a similar manner, a method for establishing wireless communication to transfer data between a patch pump and another device, for example a smartphone. To identify the patch pump to said smartphone, the patch pump and the smartphone communicate via NFC-technology in a first phase. During the communication via NFC the distance between those two devices has to be smaller due to deliberate NFC-technology design constraints. Following this first phase, in which pairing keys may have been exchanged, a Bluetooth connection using said pairing keys may be established between the patch pump and the smartphone in a second phase.

As drug delivery devices for injection usually have limited space due to their limited size and further have to comply with power, processor and further constraints, the methods known from the prior art are not satisfactory. This is particularly so because the aforementioned methods do not help to provide a simple, low-cost and reliable solution, especially one that eliminates additional load on the power source of the drug delivery device.

Further, U.S. Pat. No. 7,174,130 concerns a more secure pairing process based on the rejection of pairing keys if transmitted at high power. However, it is not disclosed how the receiving device would know what power it had been transmitted by the other device. Rather, the receiving device can measure the receiving power, which would not give this information. Thus, U.S. Pat. No. 7,174,130 proposes defaulting to a “reject all” method.

### SUMMARY

The present disclosure relates to improvements for electronic modules to be used with, or being integrated within, drug delivery devices allowing secure wireless communication between the electronic module and another device, e.g., a smart phone, a computer or a computer network, while limiting the chance of unauthorized interference by third parties.

One aspect of the disclosure relates to an electronic module suitable for use with, and/or within, a drug delivery device. Such a drug delivery device may comprise a dose setting and drive mechanism which is configured to perform a dose setting operation for setting a dose to be delivered by the drug delivery device and a dose delivery operation for delivering the set dose. The electronic module comprises at least one processor, e.g., a microcontroller, a sensor arrangement, a first communication unit with a wireless communication interface, a memory for storing measurement data, and a power source, e.g., a rechargeable or non-rechargeable battery or cell, connected to the at least one processor.

The sensor arrangement may be connected to the at least one processor and may operate to generate measurement data indicative of the dose setting operation and/or the dose delivery operation. The sensor arrangement may comprise one or more electrical switch(es) and/or may include optical and/or capacitive and/or acoustic sensors for detecting the movement of one or more component parts of the dose setting and drive mechanism of the drug delivery device. In addition, the sensor arrangement may be part of an encoding or motion sensing system.

The first communication unit may comprise a wireless communication interface connected to the at least one processor and may operate to establish an encrypted wireless communication with a second communication unit of another device such as a smartphone, a tablet, a laptop or a computer network, and to transfer data to said other device. The other device may be any other device suitable for transmitting and/or receiving information to/from said electronic module. The other device may comprise a display and/or specific software in order to display and/or evaluate said information. The wireless communication between said electronic module and said other device may be encrypted so that information being exchanged, when the electronic module is paired with said other device, it may be decrypted by the electronic module and the other device respectively. Therefore, the (wireless) communication path, e.g., the connection used to exchange information, or the information itself may be encrypted. However, the way encryption takes place is not limited by this disclosure and may be applied by common means and in common manners. One way to provide encryption may be to exchange encryption keys using the Diffie-Hellmann key exchange in order to perform the AES (Advanced Encryption Standard) encryption.

According to an aspect of the present disclosure, the electronic module may be configured to reduce the radio frequency power (RFP) of the first communication unit, while said encrypted wireless communication between said first communication unit and said second communication unit is being established, thereby limiting the distance over which the electronic module and the other device may successfully pair.

In other words, in contrast to the method proposed in U.S. Pat. No. 7,174,130 the present disclosure does not control or react to any power of the secondary device but rather controls the power of the electronic module of the pen which is sufficient to prevent a man in the middle attack.

In a model under optimized conditions, e.g., a model without sources of interference or other objects interrupting a signal, a communication unit transmits a signal approximately in a spherical shape. Thus, in a bird's-eye view, the signal spreads almost circularly, wherein the maximum outer radius determines a point at which a signal can still be received, wherein outside said radius the signal is no longer receivable. In addition, the signal can also be received within said outer radius. During pairing this maximum outer radius of transmission of a communication unit may define its pairing distance or pairing range.

Limiting the RFP of a first communication unit of an electronic module therefore limits the maximum outer radius of a signal which is transmitted by said first communication unit. To establish an encrypted wireless communication with a second communication unit of another device, said second communication unit needs to receive the pairing signal of the first communication unit and therefore needs to be within, or at the outer radius, of the pairing signal transmitted by the first communication unit. At the same time, the second communication unit needs to be able to transmit a signal which can be received by the first communication unit. Hence, during pairing, limiting the RFP of a communication unit thereby limits the pairing range, e.g., the maximum distance at which said reduced power signal, of said communication unit, may be successfully received.

As each communication unit, in order to establish an encrypted wireless communication with another communication unit, needs to transmit a pairing signal to the respective other communication unit and at the same time needs to receive a pairing signal of the respective other communication unit, limiting the pairing range of one device at the same time limits the maximum distance, e.g., the pairing distance, two communication units can be apart during the pairing process in order to successfully establish encrypted wireless communication. However, since the communication units and the respective wireless communication interfaces of said communication units are usually installed inside the devices, modules, etc., the pairing distance may also be understood as the distance between the two devices, modules etc. in such cases. In addition, transmission ranges referring to the maximum outer radius, e.g., the maximum transmission distance, may also be defined from the outer body of said devices, modules etc.

According to the previously described method by which pairing takes place, a smaller pairing distance between two communication units during the pairing process reduces the possibility of interference from an unauthorized third party, since the third party would also have to be within the pairing distance of the communication units to be able to pair with one of the respective communication units, e.g., the electronic module or the other device, in order to interfere with that pairing process during the unencrypted phase of that communication.

Further, the electronic module may comprise a feedback generator such as one or more LED(s), which may indicate dosing information, a battery charge level, and/or a pairing state, e.g., whether a pen is paired to, or in the process of pairing with, another device or not. Furthermore, such a feedback generator may indicate whether a communication was successfully encrypted or not.

In one aspect of the present disclosure, the RFP may be increased after said encrypted wireless communication between said first communication unit and said second communication unit is established, e.g., after successful pairing. Increasing the RFP allows increasing the distance between the two communication units after encrypted wireless communication has been established. Since the wireless communication is already encrypted in this phase, e.g., after the wireless communication is established, interference by an unauthorized third party, whilst not impossible, is virtually impossible due to the encryption, or at least considerably more difficult.

The increase of the RFP may be conducted stepwise so that at first the RFP may be increased to an intermediate level which may define an RFP which enables transmitting a signal in between a maximum transmission range and a pairing range. This intermediate range may be used for synchronization and thus may be called synchronization range. Also, during this synchronization stage, as in any other stage, to be able to exchange information a first communication unit and a second communication unit have to be within the synchronization range of the respective other communication unit.

However, in one example of the present disclosure, the first communication unit may increase its RFP, for example to the synchronization range, when it is paired and therefore when encrypted wireless communication has been established. While transmitting and/or receiving information with an RFP in the synchronization range, further information, which may be used for additional encryption, or information which may be more confidential than other information, may be exchanged.

It may be advantageous choosing not to use full RFP, e.g., the maximum possible range, for either of pairing or synchronization. For example, after setting the RFP during advertising, whereafter, e.g., the Bluetooth stack, is allowed to then increase power after a connection is established, which increases the robustness of the connection but maintain the desired range of interceptability.

As mentioned before, the RFP may be increased stepwise so that the RFP may also be increased to a maximum level which defines a maximum range at which a communication unit is able to successfully communicate. The maximum range may be technically constrained or limited due to further means to this maximum range. This maximum range may be called the communication range. The increase of the RFP to a level of transmission in the communication range may be conducted directly after the first communication unit is paired with a second communication unit of another device. Alternatively, the increase to said communication range may take place after a prior synchronization, wherein the communication unit is transmitting at a synchronization range. However, the communication stage may also be omitted so that after pairing the RFP may be increased to the synchronization range. The increased RFP defining the maximum range at which the communication unit is transmitting signals may also still be below the actual possible maximum range in order to reduce or limit the consumption of power from the power source, typically a rechargeable or non-rechargeable battery or cell. However, in this case, the maximum range, which is below the actual possible maximum range, indicates the distance in which the exchange of information, etc. for the set up comprising the electronic module and another device takes place.

Transmitting and/or receiving information while transmitting in the communication range may be used for communication during an exchange of data, such as data referring to dose dialing and/or dose dispensing. However, there may be no synchronization stage at all, in one aspect of the present disclosure, or the synchronization stage may be the same as the communication stage, so that the synchronization stage is the same as the communication stage. In one aspect, information may already be transmitted in the synchronization stage.

The communication unit for communicating with a communication unit of another device may comprise a wireless communication interface for communicating with another device via a wireless network such as Wi-Fi or Bluetooth®. In addition, the communication unit may comprise an interface for a wired communications link, such as a Universal Series Bus (USB) connector. For Example, the electronic system comprises a Bluetooth®, WiFi™ or similar RF unit as the communication unit. The communication unit may be provided as a wireless communication interface between the electronic module or the drug delivery device and the exterior, such as other electronic devices, e.g., mobile phones, personal computers, laptops and so on. For example, measurement data, e.g., dose data, may be transmitted by the communication unit to the external other device. The dose data may be used for a dose log or dose history established in the external device, e.g., the other device.

In one aspect of the present disclosure, the RFP, while said encrypted wireless communication between said two communication units is being established, may be reduced by software, electronic and/or mechanical means.

The reduction of the RFP may be performed using software in order to control the RFP of the communication unit(s). Further, the RFP reduction may be realized by a prior detection of a pairing process of the electronic module and a subsequent reduction of the maximum RFP of the communication unit with the help of software. In addition, an attenuator may be switched in in series with the antenna to limit the RF power radiated by the antenna, part of the power from the transmitter being absorbed by the attenuator before reaching the antenna. Thereby, the RFP of the communication unit may be indirectly limited. Further, mechanical means such as a cap or a packaging may be provided, wherein the RFP may be reduced due to the cap covering the electronic module during the pairing process. Alternatively, or additionally, a package for the electronic module and/or a drug delivery device may be provided, wherein the electronic module and/or the drug delivery device may be inserted into the package while establishing a wireless communication to another device.

Based on the foregoing description, it is clear that in the present disclosure, RFP does not represent the power actually transmitted by a communication unit, but rather the radio frequency power measurable in an environment external to the electronic module

In addition or as an alternative to the above mentioned electronic attenuation, RFP reduction could be based on switching the matching circuit to something less efficient to change the power output in a similar way and/or mechanically detuning the antenna by changing its length, or introducing shielding/absorption of some sort.

As one aspect of the present disclosure the pairing distance may be limited to be less than 3.0 meters, e.g., less than 1.0 meter, e.g., less than 0.5 meter. The pairing distance may even be 0 meter or nearly 0 meter, if the communication units or the electronic module and the other device comprising said communication units are in direct contact. As described above, the pairing distance is decisive for the risk of an unauthorized third-party interfering in the pairing process. However, even though the pairing distance as well as the transmission distance, e.g., the distance while operating in the synchronization or the communication range, should not be limited by the disclosure, in one aspect the pairing distance may be in between 0 meters and 3 meters, and the transmission distance may be in between 3 meters and up to 15 meters. The transmission distance may, however, also be up to 50 meters or even up to 100 meters with a clear line of sight. In general, the pairing distance as well as the transmission distance further depends in particular on the orientation of the devices in respect to one another as well as on possible objects which may be located within a direct line of connection between the electronic module and the other device.

For use with drug delivery devices exchanging data with a smartphone, a suitable pairing distance is e.g. 0.5 m or e.g. 1 m with both devices being on the same tabletop, whereas a suitable distance for synchronization may be above 3 m. Generally, the maximum distance is limited by the maximum transmit power set by the software in the drug delivery device and the localised environment in which the device and the other device are operated. For example, in a noisy RF environment such as close to a WiFi router the maximum distance would be lower than in a field in the countryside. Bluetooth is designed to be typically operable up to 10 m (at an RF power of typically <+10 dBm) in a typical modern environment but could potentially be 500 m line of sight in an open field at the maximum allowed RF power for a Bluetooth certified device (+20 dBm). According to the present disclosure, the device aims at maximising coin cell life and as such the RF power may be limited to give a reasonable chance of operating within a typical room not exceeding 5 m across.

The present disclosure further pertains to a drug delivery device comprising the electronic module as described above. The drug delivery device for delivery of a medicament may comprise a dose setting and drive mechanism which is configured to perform a dose setting operation for setting a dose to be delivered by the drug delivery device and a dose delivery operation for delivering the set dose and which comprises a first member. The drug delivery device may further comprise a container receptacle which is releasably attached to the dose setting and drive mechanism. As an alternative, the container receptacle may be permanently attached to the dose setting and drive mechanism. The container receptacle is adapted to receive a container, e.g., a cartridge, containing a medicament.

According to a further aspect of the present disclosure, a system comprising the electronic module as described above and another device is provided. According to one aspect at least one of the electronic module and the other device may be configured to reduce the radio frequency power (RFP) of its communication unit, e.g., the first and/or the second communication unit, while said encrypted wireless communication between said first communication unit and said second communication unit is being established thereby limiting a pairing distance between the electronic module and the other device.

As described above, the pairing range of a communication unit may be reduced by reducing its RFP. Since two devices, e.g., an electronic module and another device, may be paired if their pairing ranges overlap in such a way that they can transmit and receive information to and from each other, the pairing distance between the electronic module and another device may also be reduced by reducing the pairing range of the other device. Further, also the RFP of both devices may be reduced, thereby limiting the respective pairing ranges.

The methods mentioned above for reducing the RFP of a communication unit of an electronic module may be applied in the same way to another device. However, the methods for reducing the RFP of another device may differ from those of an electronic module, in particular to those of an electronic module for a drug delivery device, because the other device may have different capabilities and require different means due to having a different type of communication unit or that communication unit being under the control of an operating system on the other device.

In one aspect, the electronic module and the other device may be in direct contact while said encrypted wireless communication is being established. When the respective communication units of the electronic module and the other device are exposed on the outside of the module and the other device, the communication units may also be in direct contact.

Further, in one aspect the data which may be transferred between the first communication unit and the second communication unit, e.g., between the electronic module and the other device, may be encrypted. Thus, not the (wireless) communication path, e.g., the connection used to exchange information, may be encrypted but additionally or alternatively said information, e.g., said data, which is exchanged via said communication path. The keys for encrypting and decrypting a communication path and data that are transferred via said communication path may differ.

Further, in one aspect a method for operating an electronic module for a drug delivery device is part of the disclosure. The drug delivery device may comprise a dose setting and drive mechanism, which may be configured to perform a dose setting operation for setting a dose to be delivered by the drug delivery device and a dose delivery operation for delivering the set dose. The electronic module may comprise at least one processor, a sensor arrangement connected to the at least one processor and operable to generate measurement data indicative of the dose setting operation and/or the dose delivery operation, a first communication unit with a wireless communication interface connected to the at least one processor and operable to establish wireless communication with a second communication unit of another device and to transfer data to said other device, a memory for storing measurement data, and a power source connected to the at least one processor.

The aspects and features of the components of said electronic module which are already described prior to said method for operating the electronic module may also apply here.

For example, the method comprises the following steps:
a) generating measurement data indicative of the dose setting operation and/or the dose delivery operation,
b) storing said measurement data in the memory,
c) establishing encrypted wireless communication with said second communication unit of said other device by means of the first communication unit for transferring data to said other device,
d) transferring data to said other device.

In the method according to the present disclosure, while said encrypted wireless communication between said first communication unit and said second communication unit is being established, a radio frequency power of at least one of the communication units may be reduced.

In one aspect the method for operating the electronic module may further comprise the step of increasing said radio frequency power once said wireless communication between said electronic module and said other device is established.

Further, the above-mentioned step d) may comprise the step of encrypting the data transferred in said step d).

Furthermore, the above-mentioned step c) may involve exchange of encryption keys between the module and said other device.

In addition, the above-mentioned aspects of step c) may involve bringing the electronic module and said other device closer to each other than 1.0 meter, e.g. closer than 0.5 meter.

In one aspect of the present disclosure the RFP of at least one of the communication units may be reduced during the above-mentioned step c) by software, electronic and/or mechanical means.

According to a further aspect, the present disclosure is directed to a computer program adapted to execute the above-mentioned method when implemented in a processor of an electronic module, e.g., the above-mentioned electronic module, the computer program comprising computer program means for: reducing the radio frequency power (RFP) of the first communication unit while said encrypted wireless communication between said first communication unit and said second communication unit is being established thereby limiting the pairing distance between the electronic module and the other device.

In one aspect, the present disclosure is directed to a computer readable data carrier storing the above-mentioned computer program product.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omegacarboxypentadecanoyl-gamma-L-glutamyl-des (B30) human insulin (insulin degludec, Tresiba®); B29-N—(N-lithocholyl-gamma-glutamyl)-des (B30) human insulin; B29-N-(w-carboxyheptadecanoyl)-des (B30) human insulin and B29-N—(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP1 Eligen, ORMD-0901, NN-9423, NN-9709, NN-9924, NN-9926, NN-9927, Nodexen, ViadorGLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′) 2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′) 2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and equivalents thereof. An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g., the axis which extends through the proximal and distal ends of the cartridge, the cartridge holder or the drug delivery device.

### BRIEF DESCRIPTION OF DRAWINGS

Non-limiting, exemplary embodiments of the disclosure will now be described with reference to the accompanying drawings, in which:

FIG. 1 shows an embodiment of a drug delivery device comprising an electronic module according to the present disclosure.

FIG. 2 schematically illustrates an embodiment of a system comprising the electronic module according to FIG. 1 and another device.

FIG. 3 schematically shows an exemplary pairing range, synchronization range and communication range of a communication unit.

FIG. 4A schematically shows the respective ranges according to FIG. 3 of two unpaired communication units.

FIG. 4B schematically shows the communication units according to FIG. 4A during a pairing process without any third party interfering in said pairing.

FIG. 4C schematically shows the communication units according to FIG. 4A, wherein a third party is interfering in said pairing.

FIG. 4D schematically shows the communication units according to FIG. 4A during the synchronization stage.

FIG. 4E schematically shows the communication units according to FIG. 4A after being paired in a communication stage.

FIG. 5A schematically shows transmission stages and respective RFPs of communication units and a distance between said communication units throughout a communication process.

FIG. 5B schematically shows transmission stages and respective RFPs of communication units and a distance between said communication units throughout a communication process, wherein said communication units are transmitting in a pairing stage as well as in a synchronization stage.

### DETAILED DESCRIPTION

In the figures, identical elements, identically acting elements or elements of the same kind may be provided with the same reference numerals.

In the following, some embodiments will be described with reference to an insulin injection device. The present disclosure is however not limited to such application and may equally well be deployed with injection devices that are configured to eject other medicaments or drug delivery devices in general, pen-type devices, and/or injection devices.

Embodiments are provided in relation to injection devices, in particular to variable dose injection devices, which record and/or track measurement data on doses delivered thereby. These data may include the size of the selected dose and/or the size of the actually delivered dose, the time and date of administration, the duration of the administration and the like. Features described herein refer to techniques for communication of information between an electronic module and another device.

Certain embodiments in this document are illustrated with respect to the injection device disclosed in EP 2 814 545 A1 where an injection button and grip (dose setting member or dose setter) are combined. The injection button may provide the user interface member for initiating and/or performing a dose delivery operation of the drug delivery device. The grip or knob may provide the user interface member for initiating and/or performing a dose setting operation. These devices are of the dial extension type, e.g., their length increases during dose setting. Other injection devices with the same kinematic behavior of the dial extension and button during dose setting and dose expelling operational mode are known as, for example, the Kwikpen® device marketed by Eli Lilly and the Novopen® 4 device marketed by Novo Nordisk. An application of the general principles to these devices therefore appears straightforward and further explanations will be omitted. However, the general principles of the present disclosure are not limited to that kinematic behavior. Certain other embodiments may be conceived for application to injection devices as described e.g. in WO2004078239 where there are separate injection button and grip components/dose setting members. Thus, there may be two separate user interface members, one for the dose setting operation and one for the dose delivery operation.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

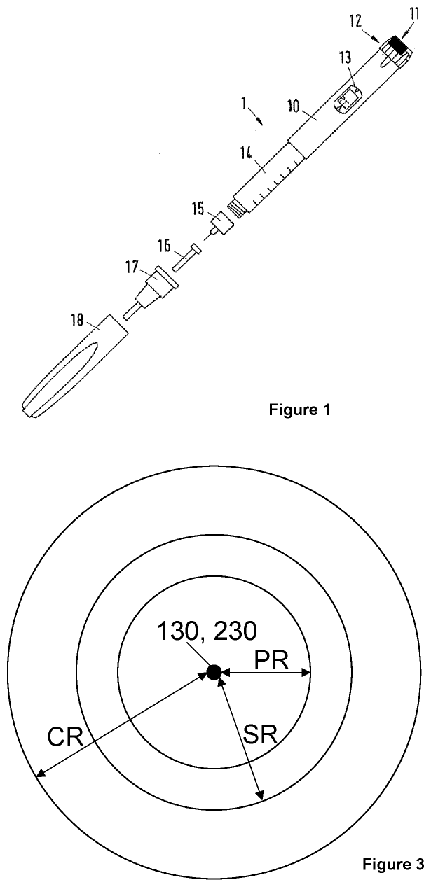

FIG. 1 is an exploded view of a medicament delivery device or drug delivery device. In this example, the medicament delivery device is an injection device 1, e.g., a pen-type injector, such as an injection pen disclosed in EP 2 814 545 A1.

The injection device 1 of FIG. 1 is an injection pen that comprises a housing 10 and contains a container 14, e.g., an insulin container, or a receptacle for such a container. The container may contain a drug. A needle 15 can be affixed to the container or the receptacle. The container may be a cartridge and the receptacle may be a cartridge holder. The needle is protected by an inner needle cap 16 and either an outer needle cap 17 or another protection cap 18. An insulin dose to be ejected from injection device 1 can be set, programmed, or ‘dialed in’ by turning a dose knob 12, and a currently programmed or set dose is then displayed via dose window 13, for instance in multiples of units. The indicia displayed in the window may be provided on a number sleeve or dial sleeve. For example, where the injection device 1 is configured to administer human insulin, the dose may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dose window 13 in FIG. 1.

The dose window 13 may be in the form of an aperture in the housing 10, which permits a user to view a limited portion of a dial sleeve assembly that is configured to move when the dial grip 12 is turned, to provide a visual indication of a currently set dose. The dial grip 12 is rotated on a helical path with respect to the housing 10 when setting a dose.

In this example, the dial grip 12 includes one or more formations to facilitate attachment of a data collection device. Especially, the dial grip 12 may be arranged to attach or integrate an electronic (button) module 11 onto the dial grip 12. As an alternative, the dial grip may comprise such a button module of an electronic system.

The injection device 1 may be configured so that turning the dial grip 12 causes a mechanical click sound to provide acoustic feedback to a user. In this embodiment, the dial grip 12 also acts as an injection button. When needle 15 is stuck into a skin portion of a patient, and then dial grip 12 and/or the attached module 11 is pushed in an axial direction, the insulin dose displayed in display window 13 will be ejected from injection device 1. When the needle 15 of injection device 1 remains for a certain time in the skin portion after the dial grip 12 is pushed, the dose is injected into the patient's body. Ejection of the insulin dose may also cause a mechanical click sound, which may be different from the sounds produced when rotating the dial grip 12 during dialing of the dose.

In this embodiment, during delivery of the insulin dose, the dial grip 12 is returned to its initial position in an axial movement, without rotation, while the dial sleeve assembly is rotated to return to its initial position, e.g., to display a dose of zero units. FIG. 1 shows the injection device 1 in this 0 U dialed condition. As noted already, the disclosure is not restricted to insulin but should encompass each drug in the drug container 14, e.g., liquid drugs or drug formulations.

Injection device 1 may be used for several injection processes until either the insulin container 14 is empty or the expiration date of the medicament in the injection device 1 (e.g., 28 days after the first use) is reached. In the case of a reusable device, it is possible to replace the insulin container.

Furthermore, before using injection device 1 for the first time, it may be necessary to perform a so-called “prime shot” to remove air from insulin container 14 and needle 15, for instance by selecting two units of insulin and pressing dial grip 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user. Nevertheless, differences (e.g., losses) between the ejected amounts and the injected doses may need to be taken into account.

As explained above, the dial grip 12 also functions as an injection button so that the same component is used for dialing/setting the dose and dispensing/delivering the dose. As an alternative (not shown), a separate injection button may be used which is axially displaceable, at least a limited distance, relative to the dial grip 12 to effect or trigger dose dispensing.

In the following, an electronic module 11 according to the present disclosure will be described with respect to exemplary embodiments and with reference to FIGS. 1 to 5B. In FIG. 1, the electronic module 11 is depicted as being integrated in the proximal end of the injection device 1, specifically integrated into the dial grip/dose button 12. As an alternative, the electronic module 11 may be a separate component part which may be permanently or releasably attached to the injection device 1, e.g., to the grip/dose button 12.

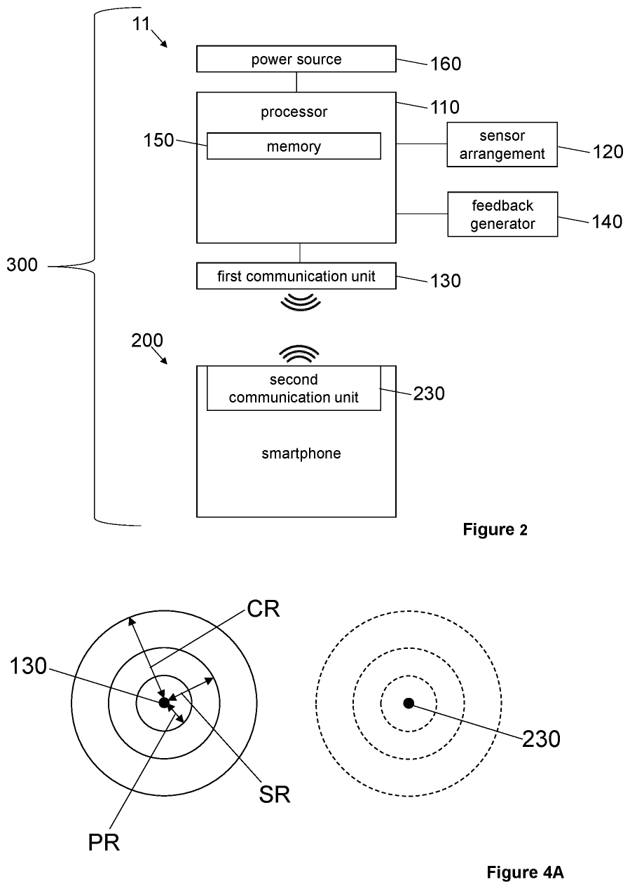

As depicted in FIG. 2, an exemplary electronic module 11 comprises a processor 110, a sensor arrangement 120, a first communication unit 130, an electronic user feedback generator 140, a memory 150, and a power source 160. In some examples, the electronic user feedback generator not necessary.

In the example depicted in FIG. 2, the sensor arrangement 120 is connected to the processor 110 and operable to generate measurement data indicative of the dose setting operation and/or the dose delivery operation. For this purpose, the sensor arrangement may comprise a LED and a photo detector together forming an optical sensor. Alternative sensor types may be implemented in addition to LED and photo detector or as an alternative thereto. Such alternative sensor types may include but are not limited to optical sensors, acoustic sensors, magnetic sensors, capacitive sensors and electrical switches.

The first communication unit 130 may comprise a wireless Bluetooth® communication interface connected to the processor 110 and operable to establish communication with another (external) device, e.g., a smartphone 200. Further, the first communication unit 130 is operable to transfer data, e.g., measurement data, to said other device 200.

The electronic user feedback generator 140 may be connected to the processor 110 and operable to generate a feedback signal to a user. In the exemplary arrangement of FIG. 2, the electronic user feedback generator 140 may comprise an LED for generating optical feedback signals. In addition to the LED, or as an alternative to the LED, the electronic user feedback generator may comprise a sounder and/or a vibration motor.

The memory 150 is adapted for storing measurement data and is connected to the processor 110 or is integrated into the processor 110.

The power source 160 is connected to the processor 110. For example, the power source 160 is a non-rechargeable, non-user replaceable coin cell.

The other device 200 depicted as a smartphone comprises a second communication unit 230. The first communication unit 130 of the electronic module 11 is configured to establish encrypted wireless communication to another communication unit, such as the second communication unit 230 of the other device 200. An established encrypted wireless communication may then be used to transmit and/or receive information and data to and from one another.

The use of an electronic module 11 with another device 200 with the aim of pairing the module and the device then may form a system 300 as shown in FIG. 2. The lines as part of a circular section shown below and above the communication units 130 and 230 illustrate a possible transmitting and/or receiving of a signal of the respective communication unit. At this point, it does not matter whether the two communication units 130 and 230 are unpaired, are in the process of being paired, are already in a synchronization stage or are in a communication stage.

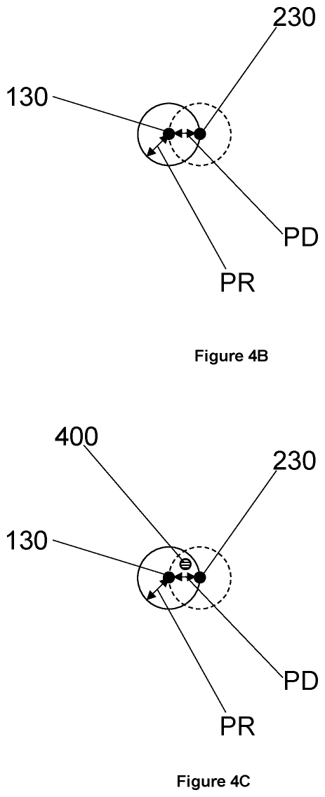

FIG. 3 schematically shows a first communication unit 130 of an electronic module 11 or a second communication 230 of another device 200 and the respective transmission ranges. The transmission ranges and the respective maximum outer radius of each range depends on a radio frequency power RFP. Therefore, the maximum transmission range in a respective stage, e.g., during a pairing stage, during a pairing process, in a synchronization stage or in a communication stage, define the maximum transmission range in a certain stage, and thus the maximum radius of successful communication in said stage. In this exemplary schematic representation, the respective maximum transmission ranges are named pairing range PR, synchronization range SR and communication range CR. However, the present disclosure is not limited to communication units having three different maximum transmission ranges and/or having different maximum transmission ranges for synchronization and communication. For example, it may be sufficient to the pairing range PR and the synchronization range SR which is also used for other communication and data transfer.

Thus, it can be noted that the pairing range PR depicts the maximum transmission range during pairing but however comprises the smallest maximum outer radius compared to the transmission ranges during synchronization stage and the communication stage. However, the relationship of the different ranges to each other is arbitrarily chosen. Nevertheless, the pairing range PR is the smallest maximum transmission range of a communication unit of an electronic module according to the disclosure and the communication range CR is the largest maximum transmission range. The synchronization range SR, if present, comprises a transmission range in between the smallest maximum transmission range during a pairing stage and the largest maximum transmission range during a communication stage. In a system comprising an electronic module and another device according to the disclosure, also the communication unit of the other device may show the aforementioned effect, wherein the pairing range PR is the smallest maximum transmission range of the communication unit of the other device and the communication range CR may be a larger or the largest maximum transmission range. In addition, the synchronization range SR, if present, may have a transmission range above the smallest maximum transmission range during a pairing stage and/or may have a transmission range identical with or below the largest maximum transmission range, e.g., during a communication stage. Thus, at least one of the two communication units being part of a system according to the disclosure comprise said reduced transmission range during pairing.

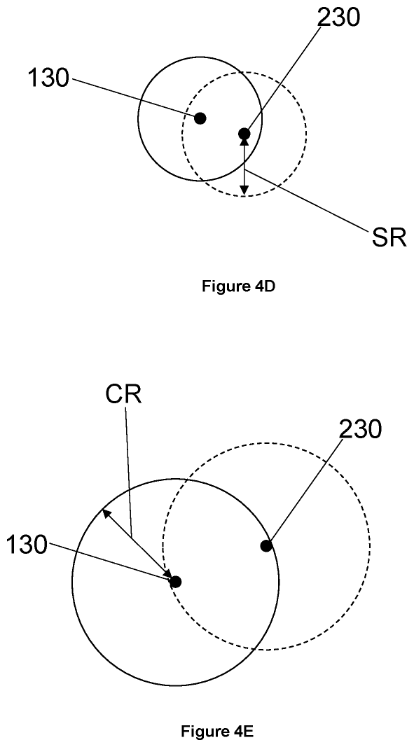

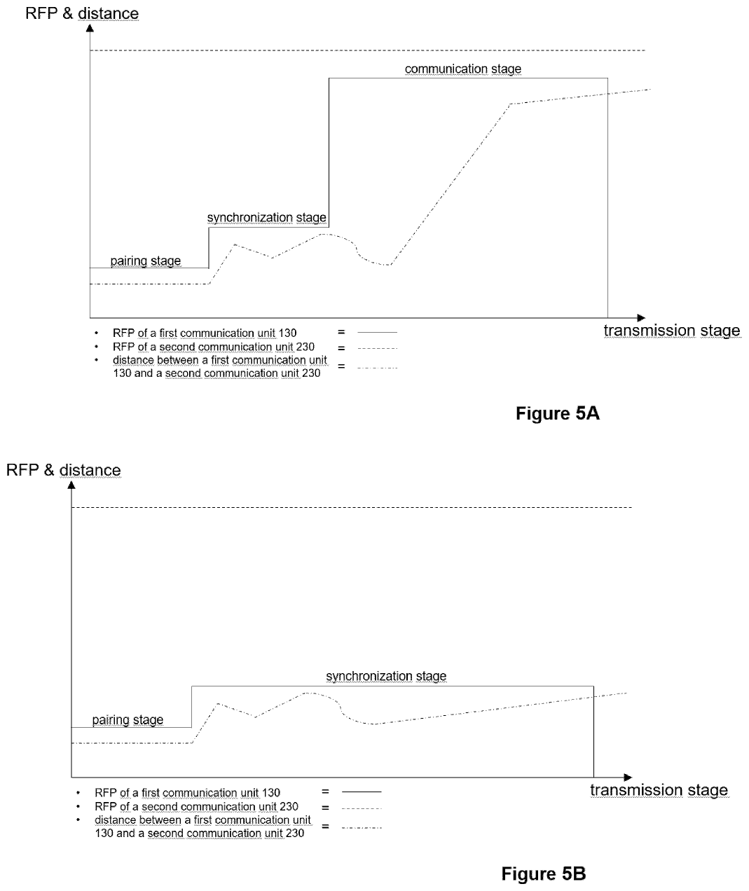

The maximum transmission range of the ranges PR, SR and CR of a first communication unit 130 may be different from those of a second communication 230 (as shown in FIGS. 5A and 5B), however they may be identically as well as shown in the following FIGS. 4A to 4E. In addition, it may be possible that a first communication unit 130 or a second communication unit 230 performs these different transmission ranges, wherein the respective other communication unit is constant transmitting at a fixed range. This fixed range may be greater than the communication range CR of one of the two communication units 130 and 230.

FIG. 4A shows the different transmission ranges PR, SR, and CR of communication units 130 and 230 before said communication units of an electronic module 11 and another device 200 are about to be paired. As mentioned previously, the transmission ranges of the two communication units 130 and 230 in this example are identical, wherein they may also be different. However, the concept of overlapping transmission ranges in a respective transmission stage (pairing stage, synchronization stage, communication stage), wherein the respective communication unit is in the transmission range of the respective other communication unit, is applied in order to have a successful pairing and a subsequent information exchange or transmission.

During the pairing process of said two communication units 130 and 230 those two communication units 130 and 230 are transmitting a respective pairing signal in a pairing range PR. The pairing ranges of the two communication units may differ, even though they are depicted identical in FIG. 4B. The smaller pairing range PR of the two communication units 130 and 230 determines the maximum allowable distance between the two communication units during the (successful) pairing process and thus the pairing distance PD.

The smaller the pairing range PR of a communication unit, the smaller the pairing distance PD between the communication unit and a respective other communication unit and thus the space in which an unauthorized third party 400 is present in order to interfere with the pairing process and thereby steal confidential data or influence the devices in other undesirable ways. The third party 400 as depicted in FIG. 4C may be a so-called ‘man-in-the-middle’, e.g., a third party in between the communication units 130 and 230. The third party 400 may be another device such as a smartphone, laptop etc. which comprises a communication unit and is able to interfere in the communication during pairing, wherein the third party device pretends to the electronic module 11, for example, the electronic module 11 of a drug delivery device, to be the other device 200 with which the electronic module 11 actually wants to connect.

After a successful pairing when encrypted wireless communication between the first communication unit 130 and the second communication unit 230 is established, the communication units may increase their transmission ranges to a synchronization range SR as shown in FIG. 4D. If one communication unit transmits at a fixed range, an increase of the radio frequency power RFP of the respective other communication unit may take place. Further, even if both communication units 130 and 230 increase their RFP the synchronization ranges SR of the two communication units 130 and 230 may differ from one another as the RFP may be increased by a different amount.

Later on, during the communication of said two communication units 130 and 230 the RFP may even be further increased to a higher level, e.g., a level at which the communication units 130 and 230 transmit in a communication range CR as depicted in FIG. 4E. If one communication unit transmits at a fixed range, an increase of the RFP as described before with respect to the synchronization range may take place in the communication unit not transmitting at said fixed range of transmission. Additionally, the RFPs of the two communication units may be increased by different amounts. While transmitting in the communication range CR, usually the relevant information such as measurement data referring to dose dialing and/or dose delivery are transferred in this so-called communication stage.

After a complete data transmission, the communication between the two communication units may be terminated even if this stage is not shown here.

FIG. 5A shows these stages, too, e.g., the pairing stage, the synchronization stage and the communication stage, wherein the synchronization stage or the communication stage are not stages that need to be mandatory as already mentioned before. Thus, there may be communication units which perform the pairing stage and the synchronization stage and there may also be communication units which perform the pairing stage and the communication stage. However, the diagram of FIG. 5A depicts exemplary the RFP of an exemplary first communication unit 130 and an exemplary second communication unit 230 of another device 200 during a pairing stage, a synchronization stage and a communication stage, wherein in contrast, FIG. 5B depicts a RFP of an exemplary first communication unit 130 and an exemplary second communication unit 230 of another device 200 which are able to perform a pairing stage and a synchronization stage. In the examples, the RFP of the second communication unit 230 is fixed throughout the entire communication between the first and the second communication unit 130 and 230. Since the RFP correlates directly and proportionally with the maximum transmission range, the RFP also reflects a maximum transmission range in the respective stages. Further, a distance between the first communication unit 130 and the second communication unit 230 during said stages is depicted. During the pairing stage said distance relates to a pairing distance PD. As can be seen, the distance between the two communication units 130 and 230 is smaller than the smallest transmission range. Here, smaller than the transmission range of the first communication unit 130. However, as depicted especially throughout the synchronization stage and in FIG. 5A the communication stage, the distance between said two communication units 130 and 230 may vary during each stage as the two communication units 130 and 230 and the respective module and the other device will not necessarily be in the same place throughout the entire communication process.

After the complete transmission of information, the first communication unit 130 in this example stops transmitting a signal in order to conserve power. The second communication unit 230 keeps transmitting a signal. From this point on, the distance between the two units 130 and 230 is irrelevant since the communication is terminated.

Finally, it should be noted that an abrupt increase in RFP and an abrupt increase in distance as shown in FIGS. 5A and B are technically impossible, therefore the diagram shows an optimized example.

As an alternative to this example, the first communication unit 130 may also transmit a signal with a fixed RFP and the second communication unit 230 may change the RFP during the communication process. As mentioned previously, a change in RFP may also take place in both communication units 130 and 230 throughout the communication process.

Hence, the present disclosure is not limited to the manner in which the RFP is changed throughout the entire communication process of a respective communication unit.

### REFERENCE NUMERALS

1 device
10 housing
11 button module
12 dial grip
13 dose window
14 container/container receptacle
15 needle
16 inner needle cap
17 outer needle cap
18 protection cap
110 processor
120 sensor arrangement
130 first communication unit
140 feedback generator
150 memory
160 power source (coin cell)
200 smartphone (other device)
230 second communication unit
300 system (electronic module and other device)
400 third party
CR communication range
PD pairing distance
PR pairing range
SR synchronization range

1-15. (canceled)

16. An electronic device for a drug delivery device comprising:
a dose setting and drive mechanism configured to perform a dose setting operation for setting a dose to be delivered by the drug delivery device and a dose delivery operation for delivering the set dose, the electronic device comprising:
at least one processor;
a memory for storing measurement data;
a power source connected to the at least one processor;
a sensor arrangement connected to the at least one processor and operable to generate measurement data indicative of the dose setting operation and/or the dose delivery operation; and
a first communication unit comprising a wireless communication interface connected to the at least one processor and configured to establish an encrypted wireless communication with a second communication unit of another device and to transfer data to the other device, wherein the electronic device is configured to reduce a radio frequency power of the first communication unit while the encrypted wireless communication between the first communication unit and the second communication unit is being established thereby limiting a pairing distance between the electronic device and the other device.

17. The electronic device of claim 16, wherein the radio frequency power is increased after the encrypted wireless communication between the first communication unit and the second communication unit is established.

18. The electronic device of claim 16, wherein the wireless communication interface is a BLUETOOTH® interface.

19. The electronic device of claim 16, wherein the radio frequency power is reduced by software operations, electronic signal, and/or a mechanical operation.

20. The electronic device of claim 16, wherein the pairing distance is limited to be less than 3.0 meters.

21. The electronic device of claim 16, wherein the power source is a rechargeable battery.

22. A system comprising:
a drug delivery device comprising:
a dose setting and drive mechanism configured to perform a dose setting operation for setting a dose to be delivered by the drug delivery device; and
a dose delivery operation for delivering the set dose; and
an electronic device configured to be coupled to the drug delivery device, the electronic device comprising:
at least one processor;
a memory coupled to the at least one processor;
a sensor arrangement connected to the at least one processor and configured to generate measurement data indicative of the dose setting operation or the dose delivery operation; and
a communication unit comprising a wireless communication interface connected to the at least one processor and configured to establish an encrypted wireless communication.

23. The system of claim 22, further comprising another device comprising another communication unit, wherein the communication unit is configured to establish the encrypted wireless communication with the other communication unit and to transfer data to the other device.

24. The system of claim 22, wherein the electronic device is configured to reduce a radio frequency power of the communication unit while the encrypted wireless communication is established.

25. The system of claim 24, wherein the radio frequency power is increased after the encrypted wireless communication is established.

26. The system of claim 22, wherein the electronic device further comprises a battery.

27. The system of claim 26, wherein the battery is a rechargeable battery.

28. A method of operating an electronic device for a drug delivery device, the method comprising:
a) generating, by a sensor arrangement of the electronic device, measurement data indicative of a dose setting operation and/or a dose delivery operation of the drug delivery device;
b) storing the measurement data in a memory device coupled to the electronic device;
c) establishing, with a first communication unit of the electronic device, a wireless connection;
d) establishing an encrypted wireless communication with a second communication unit of another device; and
e) transferring data to the other device, wherein while the encrypted wireless communication between the first communication unit and the second communication unit is being established a radio frequency power of at least one of the communication units is reduced.

29. The method of claim 28, further comprising increasing the radio frequency power once the encrypted wireless communication between the first communication unit of the electronic device and the second communication unit of the other device is established.

30. The method of claim 28, further comprising encrypting the data transferred to the other device.

31. The method of claim 28, wherein establishing the wireless connection comprises exchanging encryption keys between the electronic device and the other device.

32. The method of claim 28, wherein establishing the wireless connection comprises bringing the electronic device and the other device within 1.0 meter of each other.

33. The method of claim 28, wherein the radio frequency power of at least one of the communication units is reduced by software, an electronic signal, and/or a mechanical operation.

34. The method of claim 28, further comprising, after transferring the data to the other device, stopping transmission of data from the first communication unit to the second communication unit.

35. The method of claim 34, wherein stopping of the transmission of data from the first communication unit to the second communication unit conserves power of the drug delivery device.
